SEQUENCE LISTING (25 sequences)

SEQ ID NO: 1 - DNA, 26 bp, Artificial Sequence
  Description of Artificial Sequence pRTFor primer
tcccccgggggaagcttgcatgcctg

SEQ ID NO: 2 - DNA, 25 bp, Artificial Sequence
  Description of Artificial Sequence pRTRev primer
tcccccgggggactggattttggtt

SEQ ID NO: 3 - DNA, 30 bp, Artificial Sequence
  Description of Artificial Sequence SERK 1 Rev primer
taagtttgtcagatttccaagattactagg

SEQ ID NO: 4 - DNA, 18 bp, Artificial Sequence
  Description of Artificial Sequence AthACS marker primer 1
agaagtttagacaggtac

SEQ ID NO: 5 - DNA, 18 bp, Artificial Sequence
  Description of Artificial Sequence AthACS marker primer 2
aaatgtgcaattgccttc

SEQ ID NO: 6 - DNA, 19 bp, Artificial Sequence
  Description of Artificial Sequence AthGENEA marker primer 1
gctacgcgttgtcgtcgtg

SEQ ID NO: 7 - DNA, 21 bp, Artificial Sequence
  Description of Artificial Sequence AthGENEA marker primer 2
acataaccacaaataggggtg

SEQ ID NO: 8 - DNA, 21 bp, Artificial Sequence
  Description of Artificial Sequence nga111 marker primer 1
ctccagttggaagctaaaggg

SEQ ID NO: 9 - DNA, 21 bp, Artificial Sequence
  Description of Artificial Sequence nga111 marker primer 2
tgttttttaggacaaatggcg

SEQ ID NO: 10 - DNA, 20 bp, Artificial Sequence
  Description of Artificial Sequence nga1126 marker primer 1
cgctacgcttttcggtaaag

SEQ ID NO: 11 - DNA, 20 bp, Artificial Sequence
  Description of Artificial Sequence nga1126 marker primer 2
tcagtgcttgaggaagatat

SEQ ID NO: 12 - DNA, 20 bp, Artificial Sequence
  Description of Artificial Sequence nga1145 marker primer 1
ccttcacatccaaaacccac

SEQ ID NO: 13 - DNA, 20 bp, Artificial Sequence
  Description of Artificial Sequence nga1145 marker primer 2
gcacatacccacaaccagaa

SEQ ID NO: 14 - DNA, 20 bp, Artificial Sequence
  Description of Artificial Sequence nga162 marker primer 1
catgcaatttgcatctgagg

SEQ ID NO: 15 - DNA, 22 bp, Artificial Sequence
  Description of Artificial Sequence nga162 marker primer 2
ctctgtcactcttttcctctgg

SEQ ID NO: 16 - DNA, 20 bp, Artificial Sequence
  Description of Artificial Sequence nga12 marker primer  1
aatgttgtcctcccctcctc

SEQ ID NO: 17 - DNA, 19 bp, Artificial Sequence
  Description of Artificial Sequence nga12 marker primer 2
cttgtagatcttctgatgc

SEQ ID NO: 18 - DNA, 20 bp, Artificial Sequence
  Description of Artificial Sequence nga1111 marker primer 1
gggttcggttacaatcgtgt

SEQ ID NO: 19 - DNA, 22 bp, Artificial Sequence
  Description of Artificial Sequence nga1111 marker primer 2
agttccagattgagctttgagc

SEQ ID NO: 20 - DNA, 21 bp, Artificial Sequence
  Description of Artificial Sequence AthCTRI marker primer 1
tatcaacagaaacgcaccgag

SEQ ID NO: 21 - DNA, 20 bp, Artificial Sequence
  Description of Artificial Sequence AthCTRI marker primer 2
ccacttgtttctctctctag

SEQ ID NO: 22 - DNA, 24 bp, Artificial Sequence
  Description of Artificial Sequence AthPHYC marker primer 1
ctcagagaattcccagaaaaatct

SEQ ID NO: 23 - DNA, 24 bp, Artificial Sequence
  Description of Artificial Sequence AthPHYC marker primer 2
aaactcgagagttttgtctagatc

SEQ ID NO: 24 - DNA, 19 bp, Artificial Sequence
  Description of Artificial Sequence MBK5 marker primer 1
ctgtcagttgttggtgaag

SEQ ID NO: 25 - DNA, 19 bp, Artificial Sequence
  Description of Artificial Sequence MBK5 marker primer 2
tgagcatttcacagagacg

(19) United States
(12) Patent Application Publication
(10) Pub. No.: US 2004/0103452 A1
(43) Pub. Date: May 27, 2004

(54) Inducible apomixis

(76) Inventors: Eygeniya Russinova, Wageningen (NL); Sape Cornelis de Vries, Wageningen (NL)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/469,484

(22) PCT Filed: Apr. 9, 2002
(86) PCT No.: PCT/EP02/03958

(30) Foreign Application Priority Data
Apr. 10, 2001 (EP) 01108901.8

(51) Int. Cl. A01H 1/00; C12N 15/82

(52) U.S. Cl. 800/278; 435/468

(57) ABSTRACT

The present invention relates to vegetative reproduction or plants which is also called apomixis. In particular the invention describes a method for the production of seeds, comprising (a) transgenically expressing in the vicinity of the embryo sac of a first parent plant a gene encoding or interacting with a somatic embryogenesis receptor kinase, (b) crossing the first parent plant of step (a) with a second, genetically polymorphic parent plant and applying auxin to the crossed plants before anthesis, (c) growing F1 progeny plants from seeds obtained from the plants treated with auxin, (d) selling the F1 progeny plants obtained in step (c) to obtain F2 progeny plants and (e) selecting an F2 progeny plant which has a nuclear genome with a marker profile identical to the marker profile of the nuclear genome of the F1 progeny plant selfed in process step (d).

The present invention relates to vegetative reproduction of plants which is also called apomixis. In particular the invention describes a process step increasing the ratio of apomictic seeds formed in a plant generation, i.e. the probability of vegetative reproduction through seeds.

Apomixis is a genetically controlled reproductive mechanism of plants found in some polyploid non-cultivated plant species which results in progeny plants genetically essentially identical to the female parent plant. Genes which, upon transgenic expression in the vicinity of the embryo sac, increase the ratio of apomictic seed are described in WO 97/43427 and WO 00/24914. They either encode a Somatic Embryogenesis Receptor Kinase (SERK) or a protein interacting therewith.

Two types of apomixis, gametophytic and non-gametophytic apomixis, are distinguished. In gametophytic apomixis multiple embryo sacs typically lacking antipodal nuclei are formed or else megasporogenesis in the embryo sac takes place. In non-gametophytic apomixis, also called adventitious embryony, a somatic embryo develops directly from the cells of the embryo sac, ovary wall or integuments. Somatic embryos from surrounding cells invade the sexual ovary and one of the somatic embryos out-competes the other somatic embryos and the sexual embryo, and utilizes the produced endosperm.

Apomixis allows for true breeding, seed propagated hybrids. Thus, engineering of apomixis Into cultivated plant species will shorten and simplify the breeding process, since selfing and progeny testing to stabilize a desirable gene combination can be eliminated. Genotypes with unique gene combinations could be used as cultivars since apomictic genotypes breed true irrespective of heterozygosity. Thus, genes or groups of genes could be “pyramided and “fixed” in desired genotypes. Every superior apomictic genotype from a'sexual-apomictic cross would have the potential to be a cultivar. Apomixis engineered into cultivated plants would allow plant breeders to develop cultivars with specific stable traits for characters such as height, seed and forage quality and maturity. Breeders would not be limited in their commercial production of hybrids by (i) a cytoplasmic-nuclear interaction to produce male sterile female parents or (ii) the fertility restoring capacity of a pollinator. Almost all cross-compatible germplasm could be a potential parent to produce apomictic hybrids.

Apomixis would also simplify commercial hybrid seed production. In particular, (i) the need for physical isolation of commercial hybrid production fields would be eliminated; (ii) all available land could be used to increase hybrid seed instead of dividing space between pollinators and male sterile lines; and (iii) the need to maintain parental line seed stocks would be eliminated.

The present invention discloses a process step in the production of seeds which increases the ratio of apomictic seeds formed or developed in a plant generation transgenically expressing in the vicinity of the embryo sac a gene encoding or interacting with a somatic embryogenesis receptor kinase. According to the present invention auxin is applied to said plants before the onset of anthesis. The increased apomictic reproduction achieved can be viewed as inducible apomictic reproduction which, after withdrawal of the auxin, reverts to almost normal sexual reproduction.

The method for the production of seeds according to the present invention, comprises

(a) transgenically expressing in the vicinity of the embryo sac of a first parent plant a gene encoding or interacting with a somatic embryogenesis receptor kinase,

(b) crossing the first parent plant of step (a) with a second, genetically polymorphic parent plant and applying auxin to the crossed plants before anthesis,

(c) growing F1 progeny plants from seeds obtained from the plants treated with auxin,

(d) selfing the F1 progeny plants obtained in step (c) to obtain F2 progeny plants,

(e) selecting an F2 progeny plant which has a nuclear genome with a marker profile identical to the marker profile of the nuclear genome of the F1 progeny plant selfed in process step (d) and

f) optionally multiplying said F2 progeny plant in more than one round of selfing.

To produce a sufficient amount of seeds having nuclear genomes with identical marker profiles, apomictic plants obtained by the method described above can be multiplied in repeated cycles of selfing or crossing. For the purpose of progeny analysis it is convenient to use inbred lines in process step (b). However, the method can also be applied in situations where there is inbreeding depression.

Examples of genes to be trnsgenically expressed in the vicinity of the embryo sac are the Daucus carota SERK gene (GENBANK Accession No. U93048), the Arabidopsis thaliana SERK gene (GENBANK Accession No. A67827) as well as genes encoding proteins which physically interact with a SERK gene product such as the Arabidopsis theliana genes described by GENBANK Accession Nos. AX024556, AX024558, AX024560, AX024562, AX024564, AX024566, AX024568 and AX024570. They encode a protein having an amino acid sequence selected from the group consisting of Sequences 3 and 21 of WO 97143427 and Sequences 2, 4, 6, 8, 10, 12, 14 and 16 of WO 00/24914. Structurally related genes of similar functional and obtainable from other plant species can be used as well. To achieve expression of the transgene in the vicinity of the embryo sac the gene has to be operably linked to a cultable inducible or developmentally regulated promoter. Preferably, the gene is expressed in the female gametophyte prior to fusion of the polar nuclei with the male gamete nucleus. Of particular interest is the expression of the gene in the somatic cells of the embryo sac, ovary wall, nucellus, or integuments. Specific examples of suitable promoters are the carrot chitinase DcEP3-1 gene promoter, the Arabidopsis AtChitIV gene promoter, The Arabidopsis LTP-1 gene promoter, the Arabidopsis bel-1 gene promoter, the petunia fbp-7 gene promoter, the Arabidopsis ANT gene promoter, the Arabidopsis AtDMC1 promoter, the promoter of the O126 gene of Phalaenopsis or the SERK gene promoter.

To identify apomictic seed, the genomes of the parent plants of the initial cross ought to be sufficiently polymorphic to each other. Though, apomictic seeds are also produced, if genetically similar plants are used in the initial cross, the identification of apomictic seeds resulting from such crosses is hardly possible. Genetic polymorphisms of the parent plants, instead, allow to readily characterize the progeny plants by DNA fingerprinting and, thus, the identification of seeds resulting from apomictic reproduction. The parent plants can be considered sufficiently polymorphic, if they contain at least 5 to 10, preferably, more than 20 and even more preferably 50 to 60 or more independently segregating loci, which either show genetical variation in at least one parent plant or between the parent plants.

After the initial crossing of the parent plants auxin is applied at least once in a 1 to 10 day period before anthesis, preferably 1 to 2 days before anthesis. Repeated application of the auxin such as 2, 3, 4 or 5 times in the period before anthesis is also preferred. The auxin can be selected from the group consisting of 2,4D (2,4-dichlorophenoxyacetic acid); NAA (naphtalene acetic acid) and IAA (indole acetic acid). A particularly suitable auxin is 2,4D.

Apomictic reproduction can result in plants which are genetically identical to the female parent plant of the initial cross. Thus, within the context of the present invention the nuclear genome of the F2 progeny plant is considered essentially identical to the nuclear genome of the female parent plant used in the initial cross, i.e. process step (b) of the method described above.

The present invention can be applied to dicotylodonous and monocotyledonous plants. Among the dicotyledonous plants Arabidopsis, soybean, cotton, sugar beet, sugar cane, oilseed rape, tobacco and sunflower are preferred. Especially preferred are soybean, cotton, tobacco, sugar beet and oilseed rape. Among the monocotyledonous plants maize, sweet corn, wheat, barley, sorghum, rye, oats, turf and forage grasses, millet and rice are preferred. Especially preferred are maize, wheat, sorghum and rice.

Using sufficiently polymorphic plants for the initial cross allows to take advantage of DNA fingerprinting technologies to distinguish the various progeny plants to identify those which result from apomictic reproduction. In a specific embodiment of the method outlined above an F2 progeny plant having a nuclear genome which is essentially identical to the nuclear genome of the F1 progeny plant selfed in process step (d) above is identified by comparison of genomic fingerprints of F2 progeny plants with genomic fingerprints of the F1 progeny plants selfed to produce the F2 progeny plants in process step (d). Fingerprinting and comparing the genomes can be conveniently done using a set of molecular markers such as Restriction Fragment Length Polymorphisms (RFLPs), Random Amplified Polymorphic DNA (RAPD), Single Nucleotide Polymorphisms (SNPs), Simple Sequence Length Poliymorphisms (SSLPs), Cleaved Amplified Polymorphisms (CAPs) or Amplified Fragment Length Polymorphisms (AFLPs). A list of some specific Arabidopsis SSLPs can be accessed for example from the Arabidopsis Genome Center URL http://genome.salk.edu/. A list of Arabidopsis SSLPs and corresponding oligonucleotides can be found under the URL http://genome.salk.edu/SSLP_info/SSLPsordered.html.

Thus, the present invention further includes a method to distinguish an apomictic from a sexual progeny plant comprising characterizing the marker profile of at least 5 to 10, preferably, more than 20 and even more preferably 50 to 60 or more independently segregating molecular marker loci, in progeny and parent plants and identifying a progeny plant having a marker profile identical to the marker profile of the female parent plant, wherein the markers are polymorphic for the parent plants.

### EXAMPLES

### Example 1

### Construction of the AtLTP::AtSERK1 Expression Vector

The 2.1 kb AtSERK1 full-length cDNA is cloned as a Sacl-Kpnl fragment into the pRT105 vector (Topfer et al., Nucleic Acids Res. 15: 5890, 1987) containing the CaMV35S promoter. The CaMV35S promoter is then removed from pRT105 by Hincil-Smal digestion and replaced by the AtLTP1 (Thoma et al., Plant Physiol. 105:35, 1994) promoter fragment. The AtLTP1::AtSERK1 cassette is amplified by PCR using the following primers specific for the flanking pRT105 plasmid DNA and containing Smal restriction sites: pRTFor: 5′-TCCCCCGGGGGAAGCTTGCATGCCTG-3′ (SEQ ID NO: 1) and pRTRev: 5′-TCCCCCGGGGGACTGGATTTTGGTT-3′ (SEQ ID NO:2). The PCR fragment is then transferred into the binary vector pMOG800 (Mogen) for plant transformation after Smal digestion. The construct is verified by sequencing using the AtSERK1 specific primer SERK1 Rev: 5′-TAAGTTTGTCAGATTTCCAAGATTACTAGG-3′ (SEQ ID NO: 3) and electroporated in Agrobacterium tumefaciens strain AGL1 (Lazo et al., Biotechnology 5:963, 1991).

### Example 2

### Transformation of Arabidopsis thaliana Plants with the AtLTP::AtSERK1 Expression Vector

Arabidopsis thaliana ecotype WS plants are transformed by vacuum infiltration as described by Bechtold et al., C. R. Acad. Sci. Paris, Sciences de la vie 316: 1194, 1993). T1 seeds are selected on ½ MS-salt medium (Murashige and Skoog, Duchefa Biochemie BV) supplemented with 10% sucrose and 50mg/l kanamycine during 10 days. The kanamycine resistant seedlings are transferred into soil and used for amplification of seeds

### Example 3

### Production of Apomictic Seeds

a. Materials and Methods

Transgenic T3 Arabidopsis thaliana ecotype WS plants, i.e. transgenic plants in the third generation after transformation, that are homozygous for the AtLTP1::AtSERK1 construct are used as male donor to pollinate Arabidopsis thaliana ecotype Landsberg erecta (L r) plants. The flower buds of the F1 plants at stage 11 to 12 (Smyth et al., The Plant Cell 2: 755, 1990) approximately 1 to 2 days pre-anthesis are dipped in an aqueous solution containing 2 μM 2,4-D supplemented with 0.04% (v/v) Triton X-100 as a surfactant as described by Vivian-Smith et al., (Plant Physiol. 121: 437, 1999). The treatment is repeated twice in a two-day period and plants are left to set F2 seeds. In parallel a control cross between wild-type Ler female and wild-type WS male plant is made to obtain F1 plants. Those F1 plants are designated as wild-type F1 plants and they are analysed in the same way as the transgenic F1 plants except that they are not auxin treated.

The F2 seeds are grown into seedlings and a few rosette leaves from each plant are used for DNA extraction as described by Ponce et al., Mol. Gen. Genet. 261: 408, 1999. Simple Sequence Length Polymorphism (SSLP) analysis is carried out by simultaneous amplification of 11 SSLP markers (see Table 1) using multiplex PCR with fluorescently labeled primers as described by Ponce et al. supra. All SSLP markers used are polymorphic for WS and Ler ecotypes, homogeneously distributed in the genome and not linked in order to allow Mendelian segregation in the F2 progeny. Each forward primer is labelled with one of three different fluorescent dyes and the PCR products are separated on an ABI PRISM™ 377 DNA sequencer run in the GS 36C-2400 module. DNA fragment analysis is then performed using GeneScan® 3.1 and Genotyper® software (Applied Biosystems).

TABLE 1
SSLP markers used as described in Ponce et al. Mol Gen Genet 261: 408, 1999

| | | | | |
|---|---|---|---|---|
| SSLP | | Ler | WS | |
| name | chromosome | (bp) | (bp) | Primers |

| | | | | | |
|---|---|---|---|---|---|
| AthACS | I | 276 | 287* | 5′-AGAAGTTTAGACAGGTAC-3′ | (SEQ ID NO: 4) |
| | | | | 5′-AAATGTGCAATTGCCTTC-3′ | (SEQ ID NO: 5) |
| AthGENEA | I | 419 | 425 | 5′-GCTACGCGTTGTCGTCGTG-3′ | (SEQ ID NO: 6) |
| | | | | 5′-ACATAACCACAAATAGGGGTG-3′ | (SEQ ID NO: 7) |
| nga111 | I | 163* | 147* | 5′-CTCCAGTTGGAAGCTAAAGGG-3′ | (SEQ ID NO: 8) |
| | | | | 5′-TGTTTTTTAGGACAAATGGCG 3′ | (SEQ ID NO: 9) |
| nga1126 | II | 460 | 438 | 5′-CGCTACGCTTTTCGGTAAAG-3′ | (SEQ ID NO: 10) |
| | | | | 5′-TCAGTGCTTGAGGAAGATAT-3′ | (SEQ ID NO: 11) |
| nga1145 | II | 220 | 194 | 5′-CCTTCACATCCAAAACCCAC-3′ | (SEQ ID NO: 12) |
| | | | | 5′-GCACATACCCACAACCAGAA-3′ | (SEQ ID NO: 13) |
| nga162 | III | 88* | 86* | 5′-CATGCAATTTGCATCTGAGG-3′ | (SEQ ID NO: 14) |
| | | | | 5′-CTCTGTCACTCTTTTCCTCTGG-3′ | (SEQ ID NO: 15) |
| nga12 | IV | 252 | 262 | 5′-AATGTTGTCCTCCCCTCCTC-3′ | (SEQ ID NO: 16) |
| | | | | 5′-CTTGTAGATCTTCTGATGC-3′ | (SEQ ID NO: 17) |
| nga1111 | IV | 157 | 151 | 5′-GGGTTCGGTTACAATCGTGT-3′ | (SEQ ID NO: 18) |
| | | | | 5′-AGTTCCAGATTGAGCTTTGAGC-3′ | (SEQ ID NO: 19) |
| AthCTRI | V | 142* | 144* | 5′-TATCAACAGAAACGCACCGAG-3′ | (SEQ ID NO: 20) |
| | | | | 5′-CCACTTGTTTCTCTCTCTAG-3′ | (SEQ ID NO: 21) |
| AthPHYC | V | 226 | 211 | 5′-CTCAGAGAATTCCCAGAAAAATCT-3′ | (SEQ ID NO: 22) |
| | | | | 5′-AAACTCGAGAGTTTTGTCTAGATC-3′ | (SEQ ID NO: 23) |
| MBK5 | V | 362* | 368* | 5′-CTGTCAGTTGTTGGTGAAG-3′ | (SEQ ID NO: 24) |
| | | | | 5′-TGAGCATTTCACAGAGACG-3′ | (SEQ ID NO: 25) |

*The size of this PCR product slightly varies from the size determined by Ponce et al

b. Results/Interpretation/Discussion

We use a genetically based approach to screen for apomixis in the progeny of AtSERK1 overexpressing plants. The method is based on the use of Single Sequence Length Polymorphism (SSLP) markers. SSLPs are tandemly repeated 2 to 5 base pairs DNA core sequences. The DNA sequences flanking the repeats are generally conserved allowing the selection of PCR primers that will amplify the intervening SSLP. Variation in the number of tandem repeats results in PCR product length differences. In Arabidopsis 50 SSLP markers are described and they are conventionally used as co-dominant genetic markers for linkage and genotyping analysis. SSLPs detect a high level of allelic variation and they are easily assessable by PCR (19).

The SSLP profiles of all transgenic and control F1 plants are identical, always amplifying 22 PCR products. They corresponded to the 11 SSLP alleles in Ler and the 11 SSLP alleles in WS ecotypes that are present in all heterozygous F1 plants. F2 plants from each transgenic experiment and F2 plants from the wild-type control experiment are genotyped for the same 11 SSLP markers as described before. The SSLP profile of each F2 plant is compared with the SSLP profile of the corresponding F1 mother plant. The results are given in Tables 2 and 3.

TABLE 2
SSLP analysis on the cross between wild-type Ler and WS

| | | Plants | | | | |
|---|---|---|---|---|---|---|
| Number of | Number of | heterozygous for | | | | |
| SSLP markers | F2 plants | all SSLP markers | | χ² | | |
| scored | analyzed | Observed | Expected | (1 df) | P value | |
| 5 | 458 | 19 | 14.3 | 1.6 | | 0.2 |
| 6 | 457 | 8 | 7.21 | 0.1 | | 0.8 |
| 7 | 456 | 4 | 3.6 | 0.04 | p > | 0.9 |
| 8 | 457 | 3 | 1.8 | 0.8 | 0.4 > p > 0.3 | |
| 9 | 459 | 2 | 0.9 | 1.3 | p > | 0.3 |
| 10 | 459 | 1 | 0.45 | 0.7 | p > | 0.4 |
| 11 | 459 | 0 | 0.2 | 0.2 | | 0.7 |

TABLE 3
SSLP analysis on wild-type Ler and transgenic WS after 2, 4 D
application.

| | Total | | | | |
|---|---|---|---|---|---|
| | number of | Plants h t rozygous | | | |
| | F2 plants | for 11 SSLP markers | | χ² | |
| Type of construct | analyzed | Observed | Expected | (1 df) | P value |
| Wild-type control | 459 | 0 | 0.2 | 0.2 | 0.7 |
| (cross No 1) | | | | | |
| AtLTP::AtSERK1 | 144 | 0 | 0.1 | 0.1 | 0.8 |
| (cross No 14) | | | | | |
| AtLTP::AtSERK1 | 175 | 2 | 0.1 | 37 | <0.0001 |
| (cross No 15) | | | | | |

We scored for the number of F2 plants that are heterozygous for the 11 SSLP markers in each transgenic experiment and in the wild-type control. In a sexual population the expected number of plants that are heterozygous for 11 SSLP markers is 0.00048 meaning 1 plant out of population of 2048 plants. For the number of wild-type plants that are scored in our experiment (459 plants) the expected value is 0.2. No plants that are heterozygous for 11 SSLP markers in this population have been detected. This creates a χ2 value of 0.2, which shows that the deviation from the expected value is non-significant for the population of 459 F2 plants. These data demonstrate that wild-type Arabidopsis plants reproduce sexually. The results from the transgenic cross No. 15 show that for the AtLTP1::AtSERK1 over-expressing plants used the χ2 value is highly significant (χ2=37). In an F2 population of 175 plants we identify 2 plants that are homozygous for the 11 SSLP markers. The probability for this to happen by chance is low (p<0.0001) and we conclude that the two plants are of maternal origin and that they are apomictic progeny. In another transgenic cross (cross No. 14) with a transgenic line expressing a lower level of SERK1 protein (as detected by Western blot analysis) compared to the transgenic line used in cross No. 15 no plants heterozygous for all 11 SSLP selected markers are detected.

Based on the absence of non-reduced gametes in the transgenic plants analysed we conclude that no gametophytic type of apomixis occurred. This leaves parthenogenesis followed by dihaploidisation in the presence of fertilisation of the central cell (pseudogamy) or adventitious embryony as possible modes of apomictic embryogeneis. Since the offspring is fully heterozygous for all markers tested pseudogamy can be ruled out. This leaves adventitious embryony initiated normally before fertilisation to occur as the most likely model of apomixis.

What is claimed is:

1. A process step increasing the probability of apomixis in a plant generation transgenically expressing in the vicinity of the embryo sac a gene encoding or interacting with a somatic embryogenesis receptor kinase, wherein auxin is applied to the plants before anthesis.

2. A method for the production of seeds, comprising
(a) transgenically expressing in the vicinity of the embryo sac of a first parent plant a gene encoding or interacting with a somatic embryogenesis receptor kinase,
(b) crossing the first parent plant of step (a) with a second, genetically polymorphic parent plant and applying auxin to the crossed plants before anthesis,
(c) growing F1 progeny plants from seeds obtained from the plants treated with auxin,
(d) selfing the F1 progeny plants obtained in step (c) to obtain F2 progeny plants,
(e) selecting an F2 progeny plant which has a nuclear genome with a marker profile identical to the marker profile of the nuclear genome of the F1 progeny plant selfed in process step (d) and
(f) optionally multiplying said F2 progeny plant in more than one round of selfing.

3. The process step of claim 1 or the method of claim 2, wherein the auxin is applied at least once, preferably twice in a 1 to 2 day period before anthesis.

4. The process step of claim 1 or the method of claim 2, wherein the auxin is selected from the group consisting of 2,4D; NAA and IAA.

5. The process step or method of claim 4, wherein the auxin is 2,4D.

6. The process step of claim 1 or the method of claim 2, wherein the gene transgenically expressed encodes a protein having an amino acid sequence selected from the group consisting of Sequences 3 and 21 of WO 97/43427 and Sequences 2, 4, 6, 8, 10, 12, 14 and 16 of WO 00/24914.

7. The process step of claim 1 or the method of claim 2, wherein expression of the gene is under control of an inducible or developmentally regulated promoter.

8. The process step or method of claim 7, wherein the gene is expressed prior to fusion of the polar nuclei with the male gamete nucleus.

9. The process step or method of claim 7, wherein the gene is expressed in the somatic cells of the embryo sac, ovary wall, nucellus, or integuments.

10. The process step or method of claim 7, wherein expression of the gene is under control of the carrot chitinas DcEP3-1 gene promoter, tho Arabidopsis AtChitIV gene promoter, The Arabidopsis LTP-1 gene promoter, The Arabidopsis bel-1 gene promoter, the petunia fbp-7 gene promoter, the Arabidopsis ANT gene promoter or the promoter, the O126 gene of Phalaenopsis or the SERK gene promoter.

11.

12. The method of claim 2, wherein the marker profile of the F2 progeny plant is identical to the marker profile of the female parent plant used in process step (b).

13. The method for claim 2, wherein an F2 progeny plant having a nuclear genome with a marker profile identical to the marker profile of the nuclear genome of the F1 progeny plant selfed in process step (d) is identified after comparing genomic fingerprints of F2 progeny plants with genomic fingerprints of the F1 progeny plants selfed in process step (d).

14. The method of claim 13, wherein a set of molecular markers are used to DNA fingerprint and compare the genomes.

15. A method for the production of seeds having nuclear genomes with identical marker profiles comprising repeated cycles of selfing or crossing of plants obtained in claim 2 or the resultant progeny plants.

16. A method to distinguish an apomictic from a sexual progeny plant comprising characterizing the marker profile of at least 5 molecular markers in progeny and parent plants and identifying a progeny plant having a marker profile identical to the marker profile of the female parent plant, wherein the markers are polymorphic for the parent plants.

17. The method of claim 16 wherein the molecular markers are selected from the group consisting of Restriction Fragment Length Polymorphisms, Random Amplified Polymorphic DNA, Single Nucleotide Polymorphisms, Simple Sequence Length Polymorphisms, Cleaved Amplified Polymorphisms or Amplified Fragment Length Polymorphisms.
